(19) United States
(12) Patent Application Publication
(10) Pub. No.: US 2025/0057129 A1
(43) Pub. Date: Feb. 20, 2025

(54) AUTOMATED SYSTEM FOR BREEDING AND PROCESSING LARVAE

(71) Applicants: Kyung Kyu SONG, Jeonju-si (KR); Jin Woo KIM, Jeonju-si (KR); Jae Ha SONG, Seoul (KR)

(72) Inventors: Kyung Kyu SONG, Jeonju-si (KR); Jin Woo KIM, Jeonju-si (KR); Jae Ha SONG, Seoul (KR)

(21) Appl. No.: 18/563,634

(22) PCT Filed: May 9, 2022
(86) PCT No.: PCT/KR2022/006556

§ 371 Date: Nov. 22, 2023

(30) Foreign Application Priority Data
May 24, 2021 (KR) 10-2021-0066292

(51) Int. Cl. A01K 67/033

(52) U.S. Cl. CPC A01K 67/033; A01K 2227/706

(57) ABSTRACT

The present invention relates to an automated system in which the entire process, from breeding to processing, of breeding the larvae of an insect and at the same time sterilizing and drying the bred larvae so that the larvae can be used as an alternative raw material such as fish meal, is monitored by a management system and operated by only a small number of workforces. The automated system for breeding and processing larvae, according to the present invention, automates all of a series of processes including processing processes such as spawning, hatching, breeding, sterilization and drying of an insect, and thus simultaneously has effects such as manpower reduction, cost reduction, energy reduction, food waste treatment and securing hygiene that are required for breeding and processing and has the effect of enabling improved larvae breeding and processing.

### BACKGROUND TECHNOLOGY

Insect larvae contain abundant nutrients, including protein, and compared to the livestock industry to obtain certain nutrients, the space and breeding period required for rearing are significantly reduced, and environmental pollution due to rearing is significantly reduced compared to the livestock industry, thereby reducing human health. It is in the spotlight as a future food source, and various research results are currently being published that it is included in ingredients such as fattening feed used in livestock farming or fish farming and helps in the excellent growth of livestock or farmed fish.

In this regard, mass breeding and a thorough management system for the insect larvae are required.

However, the conventional breeding environment for insect larvae is very poor and they are not reared and processed using a factory-type automated system. Insect larvae are reared and processed in an unsanitary environment due to individuals, etc. raising and quality control of insect larvae on a small scale. It is difficult to thoroughly manage and control this, so most of the demand for the above insect larvae is dependent on imports from foreign countries.

In order to solve these structural problems, a series of processes including egg laying, hatching, rearing, sterilization, and drying of insects are automated to reduce manpower, cost, energy, and food waste disposal and processing required for breeding and processing. There is an urgent need to develop an automated system for improved larvae rearing and processing that has the effect of ensuring hygiene at the same time.

### DETAILED DESCRIPTION OF THE INVENTION

### Technical Challenges

The present invention automates a series of processes including the processing of insects, such as spawning, hatching, rearing, sterilization, and drying, to achieve effects such as reducing manpower required for breeding and processing, reducing costs, reducing energy, processing food waste, and ensuring hygiene. At the same time, by providing an improved automated system for raising and processing larvae, the breeding environment for the above-described conventional insect larvae is very poor and they are not raised and processed by a factory-type automated system, and individuals, etc. can raise and process insect larvae on a small scale. Due to quality control, insect larvae are raised and processed in an unsanitary environment, and thorough management and control of this is difficult, so we aim to solve the problem of making it impossible to raise and process large quantities of high-quality insect larvae.

### Means of Solving the Problem

In order to solve the above-mentioned problems, the present invention

A larva supply facility capable of supplying larvae of a certain size or larger to a larva farm, and the larvae supplied from the larva supply facility are placed inside a plurality of rearing cases.

A larvae rearing facility for rearing for a predetermined period of time, a stacker crane for simultaneously lifting and transporting at least one rearing case, and a predetermined amount of sweet feed using a plurality of nozzles for at least one rearing case simultaneously transported through the stacker crane. Or, an automatic feeder that feeds larvae feed, an automatic sorter that receives larvae and fecal soil reared in the larvae farm and selects them, a larva processing facility that processes the larvae selected through the automatic sorter, and the above It is possible to include an automated system for rearing and processing larvae that includes fecal soil processing equipment that processes fecal soil selected through an automatic sorter.

In this case, the larvae supply facility is a brood farm in which selected larvae composed of larvae of a predetermined size or larger are separately reared and grown into eclosion and adults, a spawning ground in which a plurality of spawning trees are graded so that adults hatched in the broiler can lay eggs, It may include a hatchery that separately separates and hatches the eggs of insects that have laid eggs in the spawning tree, and a nymph farm in which nymphs hatched in the hatchery are reared into larvae of a predetermined size, and the spawning ground is equipped with a plurality of spawning trees, and the spawning tree After insects naturally spawn on the surface, the naturally spawned eggs are separated from the surface of the spawning tree and collected, and the nymph farm is separated from the surface of the spawning tree, moved to a hatchery, and fed with nymph-specific feed to the hatched nymphs, where the larvae grow for a predetermined period of time. It is possible to grow and transport the grown larvae to a larval farm.

In addition, the larvae farm receives the larvae grown in the nymph farm so that they are distributed to a plurality of rearing cases, and then a predetermined amount of sweet feed or larvae feed is periodically introduced into the inner space of the rearing case containing the transferred larvae. Thus, the larvae can be reared automatically.

Additionally, the stacker crane is equipped with a plurality of cranes provided in the larval nursery.

Recall at least one of the breeding cases at the same time

It is transferred to an automatic feed feeder, and the automatic feed feeder includes at least one automatic feeding nozzle, and at least one automatic feeding nozzle is provided through a nozzle inlet formed in at least one breeding case transported by the stacker crane. It is desirable to simultaneously introduce and inject a predetermined amount of sweet feed or larva feed into the inner space of the breeding case.

### Effects of the Invention

The automated system for rearing and processing larvae of the present invention automates a series of processes including processing processes such as spawning, hatching, rearing, sterilization, and drying of insects, thereby reducing manpower, cost, and energy required for rearing and processing. It has the effect of enabling improved larvae rearing and processing that simultaneously has the effect of processing food waste and ensuring hygiene.

### BRIEF DESCRIPTION OF THE DRAWINGS

FIG. 1 is a schematic diagram of an automated system for rearing and processing larvae showing an embodiment of the present invention.

FIG. 2 is a perspective view of a rearing case used in an automated system for rearing and processing larvae showing an embodiment of the present invention.

### BEST MODE FOR CARRYING OUT THE INVENTION

The advantages and features of the present invention and methods for achieving them will become clear by referring to the embodiments described in detail below along with the accompanying drawings.

However, the present invention is not limited to the embodiments disclosed below and may be implemented in various different forms. The present embodiments are merely provided to ensure that the disclosure of the present invention is complete and to provide a general understanding of the technical field to which the present invention pertains. It is provided to fully inform the skilled person of the scope of the present invention, and the present invention is only defined by the scope of the claims.

The terminology used herein is for describing embodiments and is not intended to limit the invention. As used herein, singular forms also include plural forms, unless specifically stated otherwise in the context. As used in the specification, “comprises” and/or “comprising” does not exclude the presence or addition of one or more other elements in addition to the mentioned elements.

Like reference numerals refer to like elements throughout the specification, and “and/or” includes each and every combination of one or more of the referenced elements. Although “first”, “second”, etc. are used to describe various components, these components are of course not limited by these terms. These terms are merely used to distinguish one component from another. Therefore, it goes without saying that the first component mentioned below may also be a second component within the technical spirit of the present invention.

Unless otherwise defined, all terms (including technical and scientific terms) used in this specification may be used with meanings commonly understood by those skilled in the art to which the present invention pertains. Additionally, terms defined in commonly used dictionaries are not to be interpreted ideally or excessively unless clearly specifically defined.

The present invention involves rearing insect larvae, sterilizing and drying the reared larvae at the same time, and processing them into a state that can be used as an alternative raw material such as fish meal. The entire process from breeding to processing is operated solely by the monitoring personnel of the management system. This relates to an automated system in which nymphs hatch from spawned eggs, grow into larvae, and excellent individuals among the grown larvae are separately selected and repeat the process of emergence, imago, and egg production, thereby improving seeds by inducing excellent inter-individual reproduction. This relates to an automated system for larvae rearing and processing that can automate the entire process.

In addition, rather than artificially producing the thermal energy and electrical energy used in the automated system, waste heat from sweet feed and heat generated during the fermentation process are used as heat sources, and solar energy is converted into electrical energy to be used in dehumidifiers and air conditioning devices. It can be utilized as electrical energy, making it an eco-friendly automated system for raising and processing larvae with extremely low energy waste.

### Forms for Practicing the Invention

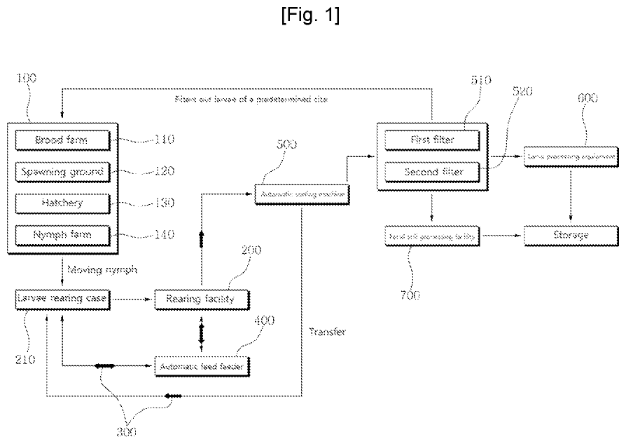

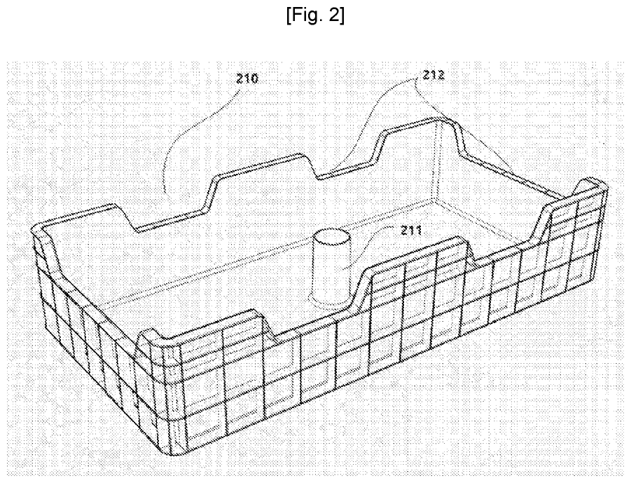

A more detailed configuration and operating principle for achieving the purpose and effect of the above-described invention will be described with reference to FIGS. 1 and 2.

FIG. 1 is a schematic diagram of an automated system for rearing and processing larvae showing an embodiment of the present invention.

Referring to FIG. 1, it is possible to check at a glance the progress of the automated system of the present invention.

More specifically, the present invention provides a larva supply facility (100) capable of supplying larvae of a predetermined size or more to a larva rearing farm within an automated system, and a larva supply facility (100) capable of supplying larvae of a predetermined size or more to a larva rearing farm within an automated system, and larvae supplied from the larva supply facility (100) within a plurality of rearing cases (210). A larva rearing facility (200) for rearing for a period of time, a stacker crane (300) for simultaneously lifting and transporting at least one rearing case (210), and at least one rearing case simultaneously transported through the stacker crane (300), an automatic feed feeder (400) that feeds a predetermined amount of sweetened feed using a plurality of nozzles, and an automatic sorting machine (500) that receives larvae and fecal soil that have been reared in the larva rearing facility (200) and selects them, including a larva processing equipment (600) that processes the larvae selected through the automatic sorting machine (500) and a fecal soil processing equipment (700) that processes the fecal soil selected through the automatic sorting machine (500). It is possible to have an automated system for rearing and processing larvae.

In this case, the larva supply facility (100) is a brood farm (110) that separately breeds selected larvae composed of larvae of a predetermined size or larger and grows them into eclosion and adults, and allows the adults hatched in the brood farm (110) to lay eggs. A spawning ground (120) with a plurality of spawning trees on a gradient, a hatchery (130) for separately hatching the eggs of insects laid on the spawning trees, and a nymph farm (140) for rearing nymphs hatched in the hatchery (130) into larvae of a predetermined size, wherein the spawning ground (120) is provided with a plurality of egg-laying trees, and after insects naturally spawn on the surface of the egg-laying tree, the naturally spawned eggs are separated and collected from the surface of the egg-laying tree, and the nymph farm (140) is separated from the surface of the spawning tree and moved to the hatchery (130), then fed to the hatched nymphs with nymph-specific feed to grow into larvae for a predetermined period of time, and the grown larvae can be transported to the nymph farm (140).

Among the larvae separated from the sorted soil through the automatic sorting machine (500), which will be described later, those that are superior in size and are larger than a predetermined size are re-introduced into the separate larva supply facility (100) to further proceed with the breeding process, such as emergence, adult larvae, breeding, and It goes through the process of spawning.

There is a feature that allows automatic seed improvement into larvae of excellent size through this process of selecting and breeding excellent individuals.

More specifically, a certain amount of pupae is input into the larva supply facility (100) every day, and the amount of light, temperature and humidity optimized for emergence are automatically controlled so that the larvae grow and pupate and go through the emergence process in brood farm (110). Equipment equipped with an automated system to naturally reproduce adult insects that have completed their breeding in the brood farm (110) and to spawn naturally in an environment as similar to nature as possible is installed on the roof or ceiling, and displays the amount of light by season and time through a separate screen, etc. Natural light that can be controlled and temperature flows in, and artificial light can be supplied when necessary.

A hatchery (130) has a spawning ground (120) equipped with spawning trees that are replaced every day, and a hatchery (130) that separates the eggs of insects laid on the spawning trees of the spawning ground (120) and hatches them by maintaining the temperature and humidity in conditions optimized for hatching. It can be equipped.

In this case, it is possible to collect and utilize the pupa shells generated in the brood farm (110) separately every day.

When transferring the laid eggs from the spawning ground (120) to the hatchery (130), it is possible to collect the laid eggs downward by shaking or shaking off the spawning tree attached to the surface.

Nymphs hatched safely in the hatching plate of the hatchery (130) cannot be reared optimally under the feed and growth conditions for rearing larvae in their current state, so the hatching plate is used as a nymph breeding box to be used as a separate box for the growth of nymphs. It is desirable to conduct breeding for a certain period of time in a space that satisfies the growth conditions.

For this purpose, optimal incubation is possible by transporting the rearing case (hatching plate) into which the nymphs are introduced to a separate nymph farm and supplying fermented sweet feed for nymphs prepared for nymphs into the rearing case into which the nymphs are introduced.

By including this optimal incubation process, there is an effect of increasing the growth rate of larvae and increasing the utilization rate of high-density breeding facilities.

In this case, it is possible to improve the breeding environment by adding fine sawdust at a predetermined ratio along with the nymphs to the nymph breeding case.

As described above, through optimal incubation, nymphs become larvae, and the rearing case in which the nymphs grow into larvae can be used as a rearing case for larvae. In this case, it is possible to supply to the breeding case not fermented sweet feed for nymphs, but general sweet feed or larva feed for larvae.

More specifically, since it has grown into a larva, the rearing case is transferred to a larva rearing facility. In this case, depending on the case, it is transferred as is on a 1:1 basis or the larvae grown in the nymph rearing case are distributed and divided into two or more larval rearing cases. It is possible to breed them.

Thereafter, it is possible to automatically rear the larvae by periodically adding a predetermined amount of sweet feed or larva feed into the inner space of the rearing case (210) containing the transferred larvae.

In this case, the stacker crane (300) simultaneously transfers at least one rearing case (210) among the plurality of rearing cases (210) provided in the larva rearing facility (200) to the automatic feed feeder (400), The automatic feed feeder (400) includes at least one automatic feeding nozzle, and feeds at least one automatic feeder through a nozzle inlet formed in at least one rearing case (210) transported by the stacker crane (300). It is preferable to inject a predetermined amount of sweet feed or larva feed into the inner space of the rearing case (210) by simultaneously inserting the nozzle.

More specifically, the rearing case (210) can be stacked in the height direction in about 10 layers to form one layer, and the rearing case (210) forming the one layer using a pallet rack structure It is possible to construct the automation system by stacking two or more layers.

The stacker crane (300) can utilize pallet racks and pallets to separate the stacked rearing case (210) of one or more layers into units and simultaneously transport and load them to achieve automation, high density, and efficiency.

FIG. 2 is a perspective view of a rearing case used in an automated system for rearing and processing larvae showing an embodiment of the present invention.

Referring to FIG. 2, if the rearing case (210) of the present invention is described in detail, the inside and outside of the ventilation hole (211) and the rearing case (210) that can induce air circulation for temperature and humidity control in order to optimize the breeding environment for larvae. It is possible to form at least one opening (212) to help air convection and to allow the nozzles of a plurality of automatic feed feeders (400) to enter the rearing case (210).

The single feed or larvae feed injected into the rearing case (210) through the nozzle of the automatic feed feeder (400) is brought in from the outside and the temperature is set to around 75 degrees Celsius and is brought into the feed tank in the automated system.

In this case, some of the sweetened feeds are improved into fermented sweetened feeds that are fermented at an appropriate temperature by mixing lactic acid bacteria, yeast, and bacillus bacteria, and the general sweetened feeds and fermented sweetened feeds are mixed in a predetermined ratio (99:1 to 98:2). It is mixed and fed at an optimal feeding temperature of around 27 degrees Celsius.

In this case, the larvae are fed several times during the growth period.

Pallets and rearing cases are automatically transported to the location where the nozzle of the automatic feeder is located by an automatic shuttle and stacker crane according to the number and order of feeding determined as described above.

Depending on the age of the larvae in each rearing case, the automatic feeder automatically feeds a set amount of feed. The pallet rack position is determined considering the next feeding order, and the pallets and rearing cases are placed in the reverse order of the above-mentioned transfer order. It is transported via stacker crane.

Additionally, the automatic sorting machine (500) transfers at least one rearing case (210) to the automatic sorting machine (500) at the same time by the stacker crane (300), after moving to the upper part of the sorting space by the conveyor and rearing case fixing device included in the automatic sorting machine (500), the rearing case (210) is rotated about a predetermined rotation axis to select the larvae and feces in the rearing case (210). It is thrown into space, in the screening space, it is possible to filter and separate the larvae and fecal soil reared for a predetermined period of time in the larvae rearing facility (200).

In this case, the rearing case (210) into which the larvae and feces have been dropped can be transported to the larva rearing facility (200) by receiving sweet feed or larval feed and larvae after undergoing a disinfection process, and the rearing case (210) is equipped with a sterilizing lamp. By controlling the amount of light and irradiation time, sterilization can be carried out and passed through the conveyor belt.

The automatic sorting machine (500) includes a first filter (510) that filters fecal soil from the larvae and fecal soil dropped into the sorting space, and it includes a second filter (520) that filters out larvae of a predetermined size or larger from among the larvae separated from the fecal soil by passing through the first filter (510), larvae of a predetermined size or larger remaining in the second filter (520) can be transferred to the brood farm (110) in the larva supply facility (100) to grow into eclosion and adults.

The automatic sorting machine (500) is separated from feces in the sorting space and washes, sterilizes and dries the selected larvae, it is possible to compress the fecal soil that has been selected and separated from the larvae in the sorting space, and inject yeast into the compressed fecal soil, package it, and then ferment it.

More specifically, larvae can be cleaned using a microbubble device or an ultrasonic cleaning device, and bacteria and germs can be sterilized through infrared rays.

In addition, the larvae that have completed the washing and sterilization process are dried to a predetermined moisture content by drying with hot air at 60 degrees Celsius and mid-infrared rays.

After the drying process, the dried larvae are processed into powder form as needed, larval oil is extracted in a milking machine, and the larva remains after milking are processed into powder form.

The larval oil and larval powder produced in the above process are stored in a low-temperature warehouse set to a predetermined temperature.

In addition, it is possible to maximize use efficiency by adding customized yeast to the fecal soil that has been separated from the larvae in an automatic sorting machine and filtered, depending on the intended use of the plant.

In this case, yeast is applied differently for each fecal soil, sealed and packaged, and then naturally fermented in a storage warehouse.

The heat energy required in the above fully automated system process is the body heat of the larvae generated by high-density rearing, waste heat from sweet feed of more than 75 degrees Celsius brought in from outside, waste heat from the washing and drying process of the larvae, and the larvae breeding farm on the first floor. An optimal eco-friendly energy system can be maintained by utilizing the convection phenomenon of high-temperature air to the second floor (nymph supply facility) and solar heat flowing in through the roof (transparent window) of a facility equipped with an automation system.

In addition, by installing a solar power generation system on the roof of a facility equipped with an automation system, it can be used as electric energy for heating and cooling of air conditioning equipment and the electric energy required to operate other automation systems.

Above, embodiments of the present invention have been described with reference to the attached drawings, but those skilled in the art will understand that the present invention can be implemented in other specific forms without changing its technical idea or essential features. You will be able to understand it.

Therefore, the embodiments described above should be understood in all respects as illustrative and not restrictive.

1. A larvae supply facility capable of supplying larvae of a predetermined size or larger to a larvae breeding farm within an automated system, comprising;
a larvae rearing facility in which the larvae supplied from the larvae supply facility are reared within a plurality of rearing cases for a predetermined period of time;
a stacker crane that simultaneously lifts and transports at least one of the rearing cases;
an automatic feed feeder that feeds a predetermined amount of sweetened feed using a plurality of nozzles to at least one breeding case simultaneously transported through the stacker crane;
an automatic sorting machine that receives larvae and fecal soil that have been reared in the larvae rearing facility and selects them;
larva processing equipment that processes the larvae selected through the automatic sorting machine; and
a fecal soil processing facility that processes the fecal soil selected through the automatic sorting machine;
includes,
the larva supply facility,
a brood farm in which selected larvae consisting of larvae of a certain size or larger are separately reared to grow into eclosion and adults;
a spawning ground with a plurality of spawning trees arranged so that the adults hatched in the brood ground can lay eggs;
a hatchery that separately separates and hatches eggs of insects that have laid eggs in the spawning tree; and
a nymph farm for rearing nymphs hatched in the hatchery into larvae of a predetermined size;
including,
the automatic sorting machine,
at least one rearing cases is simultaneously transferred to the automatic sorting machine by the stacker crane, and is moved to the upper part of the sorting space by the conveyor and breeding case fixing device included in the automatic sorting machine. After moving, the rearing cases is rotated to drop the larvae and feces in the rearing cases into the selection space,
the larvae and fecal soil reared for a predetermined period of time in the larva rearing facility are filtered and separated,
the automatic sorting machine,
a first filter that filters fecal soil from among the larvae and fecal soil dropped into the screening space; and
it includes a second filter that filters out larvae of a predetermined size or larger among the larvae that have passed through the first filter,
an automated system for raising and processing larvae, characterized in that larvae of a predetermined size or larger remaining in the second filter are transferred to the larvae in the larva supply facility to grow into eclosion and adults.

2. The larvae supply facility according to claim 1, wherein
the spawning ground is,
a plurality of egg-laying trees are provided, and after insects naturally lay eggs on the surface of the egg-laying trees, the naturally spawned eggs are separated from the surface of the egg-laying trees and collected.
a nymph farm is,
larvae are separated from the surface of the spawning tree and moved to the hatchery, then fed to the hatched nymphs with nymph-specific feed to grow into larvae within 7 days, and the grown larvae are transported to the nymph farm. Automation system for breeding and processing.

3. The larvae supply facility according to claim 1, wherein
the larva rearing facility,
the larvae grown in the nymph farm are transferred to be distributed to a plurality of rearing cases, and then a predetermined amount of single feed is periodically introduced into the inner space of the rearing case containing the transferred larvae; an automated system for rearing and processing larvae, characterized in that the larvae are automatically reared.

4. The larvae supply facility according to claim 1, wherein
the stacker crane,
at least one rearing case among the plurality of rearing cases provided in the larva rearing facility is simultaneously transferred to the automatic feed feeder,
the automatic feed feeder,
it includes at least one automatic feeding nozzle, and at least one automatic feeding nozzle is simultaneously introduced through a nozzle inlet formed in at least one rearing cases transported by the stacker crane to feed the rearing cases; an automated system for rearing and processing larvae, characterized by injecting a predetermined amount of sweet feed into the internal space.

5. The larvae supply facility according to claim 1, wherein
the rearing cases into which the larvae and feces were dropped,
an automated system for raising and processing larvae, characterized in that it is transported to a disinfection facility by the stacker crane, undergoes a disinfection process, receives sweet feed and larvae, and is then transported to a larva rearing facility.

6. The larvae supply facility according to claim 1, wherein
the automatic sorting machine,
an automated system for rearing and processing larvae, which is characterized by washing, sterilizing, and drying the selected larvae separately from fecal soil in a sorting space.

7. The larvae supply facility according claim 1, wherein
the automatic sorting machine,
an automated system for rearing and processing larvae, which is characterized in that fecal soil is separated from larvae in a sorting space and selected, compressed fecal soil, yeast is added to the compressed fecal soil, packaged, and then fermented.
